(19) United States
(12) Patent Application Publication
(10) Pub. No.: US 2003/0194718 A1
(43) Pub. Date: Oct. 16, 2003

(54) Probing sequence determination system for DNA arrays

(75) Inventors: Hiroyuki Tomita, Tachikawa (JP); Toshiro Saito, Hatoyama (JP); Masatoshi Narahara, Sayama (JP); Hirokazu Kato, Hatoyama (JP)

Correspondence Address: Stanley P. Fisher, Reed Smith Hazel & Thomas LLP, Suite 1400, 3110 Fairview Park Drive, Falls Church, VA, 22042-4503, US

(73) Assignee: Hitachi. Ltd.

(21) Appl. No.: 10/229,058

(22) Filed: Aug. 28, 2002

(30) Foreign Application Priority Data
Apr. 15, 2002 (JP) 2002-111490

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

It is needed to implement probing sequences having no potentiality of cross-hybridization, equal Tms, and less secondary structures for the probing sequences for DNA arrays so that the sensitivity and repeatability of measurement may be ensured. The probing sequences for the DNA arrays can be determined by following at least two steps, one being a step for creating a list of candidate probes for the DNA probe arrays, which are made of partial sequences of genome or cDNA sequences (the list-up step) and the other being a step for discarding the candidate probes, of which melting temperatures deviate from a given range, which have higher stability of secondary structures, or which have the potentiality of cross-hybridization, from the primary candidate probe list (the filtering step). The present invention thus provides a method for designing probing DNA sequences to be fixed on the DNA probe arrays.

### BACKGROUND OF THE INVENTION

1. Field of the Invention

The present invention relates to a method for designing DNA probing sequences for DNA probe arrays or devices called DNA chips, which are intended to measure gene expression distributions in physiological samples.

2. Description of the Related Art

Genetic information described in genes on DNA molecules in vivo is transcribed onto RNAs and translated into proteins in ribosome. A process involving the transcription onto the RNAs and the translation into proteins is referred to as gene expression. It has been known that in various types of activities, such as cell division, homeostasis, responses to external stimuli, aging, and apoptosis, different gene expression distributions are exhibited. It may be said that the above-mentioned gene expression distributions also vary depending on, for example, the presence of any disease and its malignancy. Abnormalities in more than one genes contribute to life-style related diseases and multifactorial disorders, such as allergic and stress diseases, except for some diseases such as hereditary diseases. This suggests that to diagnose these diseases, it is required to detect abnormalities in more than one gene at the same time. It is considered that in many cases, any abnormalities are reflected as variations in gene representation, for example, increase or decrease in gene representation. This means that a technique for viewing more than one gene representation distribution is useful and essential for diagnosis of the diseases and the like. A technique called the DNA array method (Duggan, D. J. et al., Expression profiling using cDNA microarrays, Nature genetics supplement, vol. 21, p10-14, 1999), which allows the variations in representation of several hundreds to several tens thousands genes to be viewed at the same time, provides us with more than one gene expression distribution, being superior to other techniques, for example, in diagnosing the multifunctional diseases. In addition, this technique has higher practicability because it can achieve convenience in measurement, high throughput, and low cost all together.

In general, a DNA array is fixed on a glass slide using a complementary DNA (cDNA) having a messenger RNA-complementary sequence as a probe (Lockhart, D. J. et al., Expression monitoring by hybridization to high-density oligonucleotide arrays, Nature biotechnology, vol. 14, p.1675-1680, 1996). An experimenter takes the messenger RNAs out from cells, organs, or others and then synthesizes the fluorescence-labeled cDNAs from the synthesized messenger RNAs (hereafter, simply referred to as mRNAs) using reverse transcriptase and a fluorescence labeling material. Then, the fluorescence-labeled cDNAs are applied on the DNA array. Next, the DNA array is left at a given temperature for a given period to hybridize the fluorescence-labeled cDNAs and the DNA array. Next, the fluorescence-labeled cDNAs nonspecifically hybridized on the DNA array are washed away from it (the nonspecifically hybridized cDNAs have weaker complementary bonding force than the specifically hybridized cDNAs have). Finally, an intensity of a fluorescent signal corresponding to each of probes fixed on the DNA array is measured. The number of mRNAs is proportional to the intensity of the hybridized fluorescent signal corresponding to each of the probes fixed on the DNA array. In this way, the number of mRNAs corresponding to various types of genes in the organs or the cells can be measured. For example, mRNAs can be taken out from the cells or the organs to at each of given time points lapsed after the administration of a drug and the number of mRNAs is measured to observe a time-varying transition in the number of mRNAs for each of genes. An increase in the number of mRNAs means that genetic codes are actively transcribed from the DNA molecules, which explains that behavior of genes enhance. An analysis of the time-varying transition in the behavior of genes will give information on any abnormalities in genes distributing to diseases.

### SUMMARY OF THE INVENTION

A DNA array is an advantageous method in that it allows more than one gene expression distribution to be easily measured with no difficulty at a same time, although its repeatability of measurement and its measurable minimum value have not yet reached a satisfactory level compared with other medical equipment, for example, biochemical or immune analytical equipment. Giving an example, the repeatability of measurement for biochemical or immune analytical equipment is at a level of 1±0.05-fold (5% of CV value), while that for the DNA array is at a level of approx. 2-fold (or ½-fold, 100% of CV value). As one of reasons for its low repeatability, cross-hybridization is assumed. Cross-hybridization is such a phenomenon that many-to-one association is established between the mRNAs or the cDNAs from a sample and DNA probes fixed on the base plate though one-to-one association is to be inherently observed between them. Since even mRNAs derived from different genes have sequences analogous each other (for example, individual genes belonging to a same super family have many analogous sequence sites), the probes containing these analogous sequence sites may be hybridized, for example, with the mRNAs derived from many genes belonging to the same super family. For this reason, when expression strengths of genes having analogous sequences are different from each other even if the expression strengths of genes to be measured are identical, the expression strengths of genes to be measured are measured mistakenly assuming that they changed, leading to deterioration in the repeatability of measurement. Alternately, in many cases, a portion of a sequence of a cloning vector may be used as a primer (hereafter, simply referred to as a common primer) in amplifying the probing DNA from the cDNA inserted into any of the cloning vectors, such as pCU18, 19 and M13mp18, by the Polymerase Chain Reaction (PCR) method. The amplification of the cDNA (containing approx. 1000-2000 bases on average) inserted into the cloning vector using the common primer will give a total length of inserted cDNA including the analogous sequence. When the inserted cDNA is fixed on the base plate, cross-hybridization may occur because, for example, more than one gene having many analogous sequence sites and belonging to the same super family is hybridized. For this reason, the probing DNAs must be so arrayed and designed that no cross-hybridization may occur.

Further, to achieve high-accuracy (or high-stringency) of hybridization between each of DNA fragments fixed on the DNA array and each of sample-derived DNA fragments, a relationship between hybridization temperatures (Th) and melting temperatures (Tm) of the fixed DNA fragments must not be bypasses. That is because it is known that the following relationship is established between sequence homology among the DNAs and the Tms. In other words, it is such a relationship that a mismatch rate of 1% between two DNAs drops the Tms of the DNAs by approx. 1.4° C. This means that the sample-derived DNA fragment, which has a match rate of 100% (an identity of 100%) with the base sequence of the fixed DNA fragment, is hybridized when the Tm of fixed DNA fragment is equal to the Th. Note that the identity is a value indicating a percentage of the match rate between two DNAs to be compared and the identity is defined to be 100% when all the corresponding bases of two DNAs are identical. If a difference between the Th and the Tm is 10° C., the sample-derived DNA fragment having a match rate of approx. 93-100% with the base sequence of the fixed DNA fragment is hybridized based on a formula, 100%−(10° C./1.4° C.)=92.9%. Similarly, it may be expected that when the difference between the Th and the Tm is 20° C., the DNA fragments having an identity of 85.7-100% are hybridized and when the difference between them is 30° C., the DNA fragments having a sequence match of 78.6-100% are hybridized. To conduct high-accuracy separation, it is desirable that only the DNA fragments having a match rate of, for example, at least 80-100% are hybridized. It can be said that any difference of 30° C. or lower between the Th and the Tm will result in a target value for the probe Tm (in many cases, a total average of the Tms for all the probes) ±15° C. or lower. Thus, to achieve high-accuracy hybridization, it is requisite that the difference between the melting temperatures of the fixed DNA fragments and the hybridization temperature is taken into account. Hereafter, the DNA fragments fixed on the DNA probe array are referred to as probing DNAs. The cDNA synthesized from the messenger RNA (mRNA) taken out from the organ or the cell using reverse transcriptase or the mRNA taken out from the cell are simply referred to as a target. Sometimes, they are referred to as a target DNA and a target RNA, respectively. The target is often labeled using, for example, fluorescent labeling but labeling on the target is not always needed. Hereafter, throughout this specification, it is assumed that the DNA is used as the target, although the probing DNA array can be designed according to the method disclosed in the specification of the present application even tough the target is the RNA.

The probing DNA hybridizes with the target in the form of single-strand DNA. On the other hand, the single-strand DNA has a higher-order structure formed through its own hydrogen bond in a solution. This higher-order structure is referred to as a secondary structure. Probe-target hybridization is a process, which competes with the formation of secondary structure. This means that when the concentrations of the targets are identical, the frequency of probe-target hybridization increases with lower stability of the secondary structure, enhancing the signal strength in hybridization. For this reason, it is desirable that the stability of the secondary structure of the probing DNA is low.

As shown from the above explanations, it is necessary for the probing DNAs to be used for the DNA probing array to have higher their own specificities, have almost equal Tms, and have low potentiality for having the secondary structure. In the following paragraphs of the specification of the present application, a method for implementing the probing sequence, in which the probes fixed on the DNA array have higher their own specificities, have almost equal Tms, and have low potentiality for having the secondary structure, is described.

### BRIEF DESCRIPTION OF THE DRAWINGS

FIG. 1 is a view showing an example flowchart, which explains the whole structure of the probe design process.

FIG. 2 is a view showing an example flowchart, which explains the whole structure of the probe design process.

FIG. 3 is a view showing an example flowchart, which explains the list-up step.

FIG. 4 is a view showing an example flowchart, which explains the filtering step.

FIG. 5 is a view showing an example flowchart, which explains the re-examination step.

FIG. 6 is a list showing oligo-DNA sequences used in the experiment and the identities.

FIG. 7 is a list showing the correspondence between the identities and target-derived signals or non-labeled target-derived signals.

FIG. 8 is a view showing an example flowchart, which explains the whole structure of the probe design process.

FIG. 9 is a view showing a genome/cDNA sequence file.

FIG. 10 is a view showing an example file containing motif information.

FIG. 11 is a view showing an example file containing the parameters used in thermodynamic computation.

FIG. 12 is a view showing an example file containing the patterns, against which the patterns are matched.

FIG. 13 is a view showing an example file containing repetitive sequences.

FIG. 14 is a list showing the secondary candidates for probes.

FIG. 15 is a view showing an example of a probe design system interface.

FIG. 16 is a view showing the melting temperatures of the probes.

FIG. 17 is a view showing the match rates with the most-analogous gene fragments focusing on the potentiality of probing cross-hybridization.

FIG. 18 is a view showing the differences in melting temperature from the most-analogous gene fragments focusing on the potentiality of probe cross-hybridization.

FIG. 19 is a view showing the differences in free energy level from the most-analogous gene fragments.

FIG. 20 is a view showing an example interface, which explains the whole process.

### DETAILED DESCRIPTION OF THE INVENTION

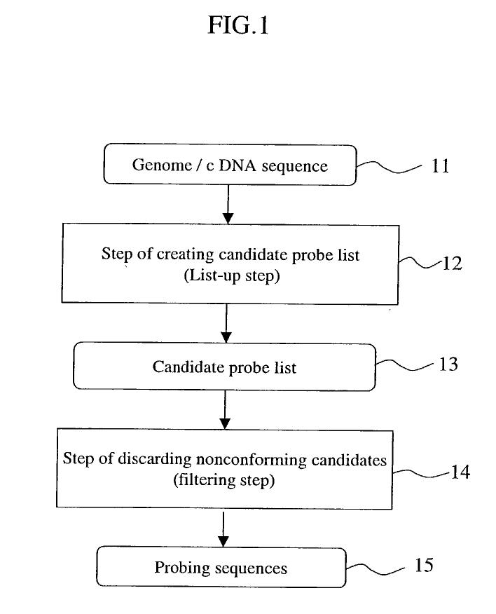

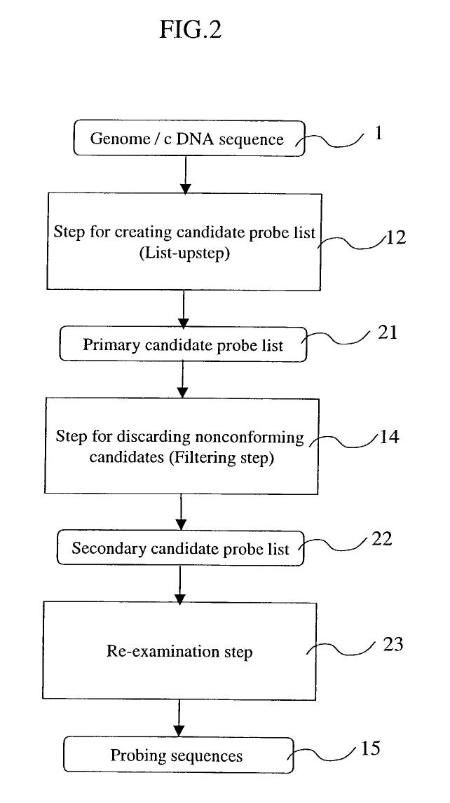

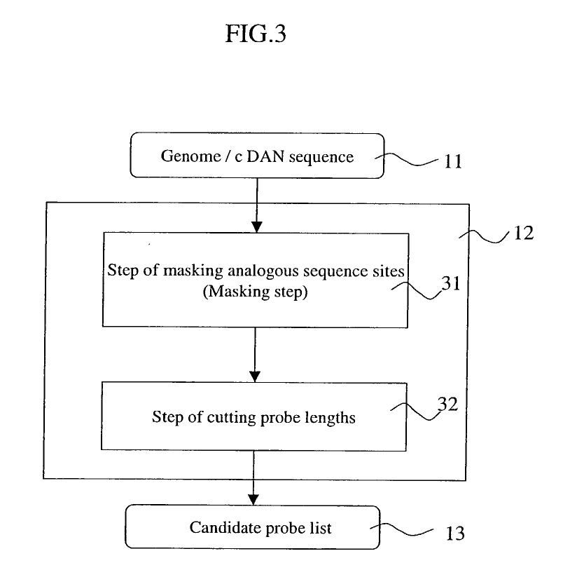

To design probing sequences for DNA arrays, first, a genome DNA sequence or a messenger RNA (mRNA) sequence must be obtained from public or commercial databases including base sequence databases such as GENBANK and the Expressed Sequence Tag (EST) sequence database. Instead of the messenger RNA, a cDNA sequence, a sequence complementary to the mRNA sequence, may be obtained because in many cases, the probes are made of the DNA. The obtained genome DNA or cDNA sequence is used at a starting point in the design process as shown in FIG. 1. A partial sequence on the genome/cDNA sequence corresponds to a desired probing sequence 15. As described in SUMMARY OF THE INVENTION, the probing sequence 15 is the partial sequence of a genome/cDNA sequence 11 and is characterized by low homology with other gene sequences, its Tm falling within a given range, and low potentiality of having the secondary structure. Hereafter, the process of determining the probing sequence 15, which have these characteristics, from the genome/cDNA sequence 11 is referred to as the probe design. In brief, all the partial sequences of the genome/cDNA sequence 11 can serve as the probes. Hereafter, the partial sequences of the genome/cDNA sequence 11, which can serve as the probes, are referred to as candidate the probes. All the candidate probes, however, are not always suited for the probes. That is because some of candidate probes may have higher homology to other gene sequences, their Tm deviated from the given range, and/or high potentiality of having the secondary structure. For this reason, in the flow of the probe design process, it is desirable to undergo two steps, one being a step 12 of creating a candidate probe list (a list-up step), in which the candidate probes are listed, and the other being a step 14 of excluding nonconforming candidates from the candidate probe list 13 created in the previous step 12 (a filtering step). If the list-up step 12 and the filtering step 14 are combined into one step, the melting temperature, the presence of the secondary structure, and the potentiality of cross-hybridization must be checked for all the partial sequences of the genome/cDNA sequence 11. However, simply by examining the candidate probe list 13 created in the list-up step 12, the melting temperature, the presence of the secondary structure, and the potentiality of cross-hybridization can be easily checked, resulting in reduction in computation time. Only probe sequences, which passed the filtering step 14, may be the probing sequences 15 to be used for the DNA arrays. A simplest probe design flow is shown in FIG. 1, but if the filtering step 14 takes a long time, the probing sequence 15 may be synthesized by following a step 23 of conducting a re-examination of a primary candidate probe list 21 created in the list-up step 12 and a secondary candidate probe list 22 created in the filtering step 14 (a re-examination step) as shown in FIG. 2. The advantage of probe design according to the sequence in FIG. 2 is in that the time required for probe design can be reduced by combining a series of steps from the filtering step 14 to a step of determining the probing sequence 15 into two steps, the filtering atop 14 and the re-examination step 23, for example, to assign the filtering step 14 to a computer A and the re-examination step 23 to a computer B for independent processing. Note that the primary candidate probe list 21 shown in FIG. 2 is the same as the candidate probe list 13 shown in shown in FIG. 1, although a different name is used in FIG. 2 to emphasize that it consists of two sub-lists. As shown in FIG. 3, the list-up step 12 can be divided into a step of excluding analogous sequence sites 31 and a step of cutting out a given length of probes 32. In the step of excluding analogous sequence sites 31, the candidate probes containing the sites having sequences analogous to other gene sequences, for example a motif site and a repetitive sequence site, are discarded from the candidate probe list 13 before the candidate probe list 13 is created. This step is also called a masking step 31 because the sites having the sequences analogous to other gene sequences are masked so as not to use as the probes. The masking step 31 is not always needed in designing the probes but it is recommended that if possible, the masking step 31 is incorporated in the design process because the filtering step 14 can be undergone faster by excluding the sequences analogous to other gene sequences from the candidate probe list 13.

Note that usually, motifs mean elements of a structure or a pattern but in this specification, they indicate given structures found in amino-acid sequences of various kinds of proteins. The motifs are the structures commonly found in a wide range of proteins having different functions. Note that the protein has structures, called domains, made of various combinations of motifs. In the specification of the present application, the motifs indicate sub-units smaller than the domains of the protein. The motifs include, for example, DNA-binding structural modules called a helix-turn-helix or a zinc finger.

The step 32 following the masking step 31 involves obtaining (cutting out) the candidate probes, which are partial sequences of a base pair with a length of L2, from the genome/cDNA sequence of the base pair with a length of L1. For example, when L1=1000 base pairs and L2=100 base pairs, 901 candidate probes may be cut out (L1−L2+1=1000−100+1=901). Note that in this example process, the masking step 31 is bypassed for simplification. The list of these 901 partial sequences, namely candidate probes, is created in the step 32 of cutting out a given length of probes.

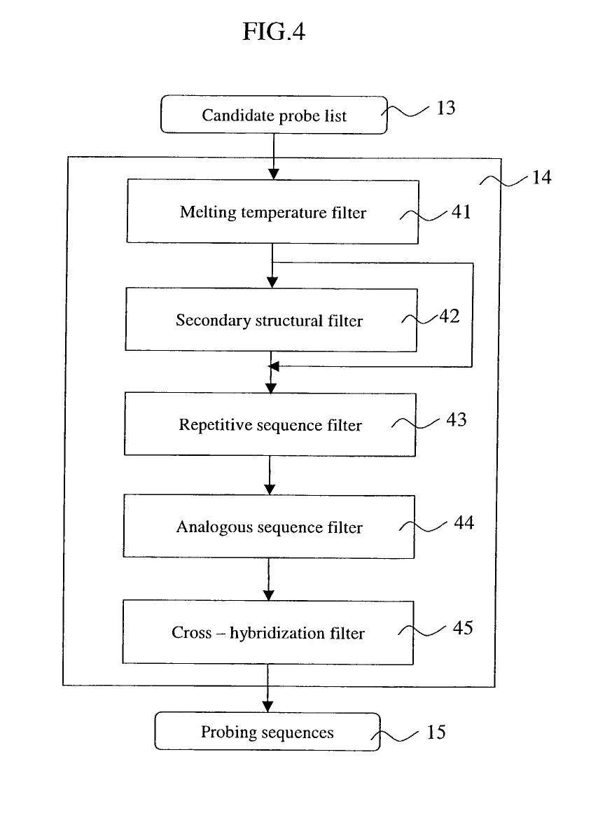

As shown in FIG. 4, the filtering step 14 can be divided into more than one sub-step. These sub-steps of the filtering step 14 correspond to the individual characteristics essential for the probing sequence 15. The characteristics essential for the probing sequence 15 includes: for example, (1) its melting temperature (Tm) falling within a given range, (2) its single-strand DNA probes having no unstable secondary structure, (3) it having no repetitive sequence prone to develop a nonspecific sequence, (4) it having no analogous sequence with high homology to other gene sequences, and (5) no potentiality of cross-hybridization with other gene sequences. For this reason, the filtering step 14 may involve, for example, five sub-steps, namely a melting temperature filter 41, a secondary structural filter 42, a repetitive sequence filter 43, an analogous sequence filter 44, and a cross-hybridization filter 45, each of which corresponds to one of the above-mentioned characteristics (1)-(5). The melting temperature filter 41 is used to determine whether the melting temperatures of individual candidate probes registered in the candidate probe list 13 fall within a predefined range of melting temperatures estimated from the sequences and go to the next step if so, while discard them from the candidate probe list 13 if not so.

The secondary structural filter 42 is used to estimate the stability of the secondary structure in a solution (free energy ΔG (the whole secondary structure)), compare the estimated stability with the inherently desired stability of probe-target hybridization (free energy ΔG (probe-target hybridization)), and go to the next step if the stability of the secondary structure is sufficiently low, while discard the candidate probe from the candidate probe list 13 if the stability of the secondary structure exceeds a given threshold and the secondary structure is kept stable. The sufficiently low stability of secondary structure means, for example, the following formula 1 is satisfied. ΔG (the whole secondary structure)<α×ΔG (probe-target hybridization)

(Formula 1)

Where, α is, for example, any of 0.01-0.5.

The Nussinov algorism or the Zuker algorithm (R. Durbin et al., Biological Sequence Analysis, Cambridge University Press, Chapter 10, 1998), for example, may be used for predicting secondary structures. The fundamental principle of forecasting secondary structures is to minimize structural energy. The minimization of structural energy can be assessed depending on the magnitude of a molecular partition function, q, one of Boltzmann functions described in the formula 2.

q=Σgi*exp(−εi/kT) (Formula 2)

Where,

εi=i^{th} energy level,

gi=energy of εi,

k=a Boltzman constant, and

T=an absolute temperature.

To minimize energy of the secondary structure directly from the formula 2, a considerable computation time is required. To overcome this problem, the Zuker method shown in the formulas 3-5 is used for reducing the computation time. The whole secondary structure S has such a structure that the DNA or RNA is uniquely looped. This can be represented by the following formula 3.

R=∪L(i,j) i,j∈S (Formula 3)

Where,

R a base pair set,

i,j=two base pairs, and

S=the whole secondary structure.

The DNA and RNA complementarily bind to form a double-strand and enter the stable state (free energy decreases), while on the contrary, a single-strand formed by the secondary structure enters the unstable state. This destabilization can be differently said to be increase in free energy. An increase in free energy δδG can be represented by the following formula 4 when the base length ls of the single-strand portion is less than 30 base pairs and by the following formula 5 when it is equal to or more than 30 base pairs.

δδG=1.75*RT*ln(ls) (Formula 4)

Where,

δδG==an increase in free energy depending on the loop structure,

R=a gas constant,

T=an absolute temperature, and

ls=a length of the single-strand portion, namely the number of bases therein.

δδG=δδG30+1.75*RT*ln(ls/30) (Formula 5)

Free energy of the secondary structure ΔG (the whole secondary structure) can be calculated by summing stabilization energy of the double-strand portion ΔG (a double-strand portion) and destabilization energy of the single-strand portion δδG as shown below.

ΔG(the whole secondary structure)=ΔG(a double-strand portion)+δδG (Formula 6)

Note that the step of the secondary structural filter 42 is desirably followed when the probing DNAs fixed on the base plate are single-strand DNAs, while this step may be bypassed when the probing DNAs fixed on the base plate are double-strand DNAs. That is because it is difficult to estimate the stability of the secondary structure for the reason that the probing DNAs may be in the equilibrium state in a solution, which allows them to form double-strands or loose the portions of the strands into single-stands. Further, it is desirable that a mini-hairpin sequence can be used as the secondary structural filter 42 to search for the candidate probes having the mini-hairpin sequence, which are to be discarded from the candidate probe list 13. The mini-hairpin indicates a short sequence having high stability (its melting point exceeds 70° C.), for example, GCGAAGC (the sequence 1) or GCGAAAGC (the sequence 2). It can be expected that Prove-target hybridization can be inhibited because the portion of min-hairpin acts as a 3D obstacle. A special attention must be paid in assessing these mini-hairpin sequences only using the Nussinov algorithm or the Zuker algorithm because they may be often missed.

The next step of the repetitive sequence filter 43 is used to discard from the candidate probe list 13 the candidate probes having repetitive sequences including a two base-repeat, namely the sequence in which two bases are repeated, for example, AT or GC, a three-base repeat, for example a CXG repeat (X indicates any of A, T, G, and C), or a human Alu repetitive sequence. The site containing a repetitive sequence has higher potentiality of cross-hybridization because it may hybridize with the target derived from other gene including the same repetitive sequence. On the other hand, the two-base repeat or the three-base repeat may cause the probe selectivity to deteriorate because they allow the same sequences to be retained even if a shift of two or three bases occurs.

The next step of the analogous sequence filter 44 is used to search the candidate probes for any sequences analogous to other gene sequences by comparing them and discard the candidate probes having the sequence analogous to other gene sequences, if any, from the candidate probe list 13. The step of comparing the sequences between the probes and the other genes is called homology search and any of heuristic algorithms, such as the blast algorithm and the FASTA algorithm, may be used, alternately any of local alignment algorithms, such as the Smith-Waterman method, or any of algorithms using the Hidden Markov model may be used. Generally, the analogous sequence filter 44 is the most time-consuming step in the probe design process because the size of a gene sequence database is huge. To improve the speed of computation, strings may be converted into numeric characters to execute, for example, bit-shift computation.

The last sub-step of the filtering step 14 is a cross-hybridization filter step 45. A difference between the cross-hybridization filter step 45 and the analogous sequence filter step 44 is described below. The analogy of the probing sequences to the other gene sequences resulted from the comparison using the analogous sequence filter 44 can be represented by an expected value E or a statistically significant level (p value). For example, according to the Karlin and Altschul formulae, the p value can be obtained by the formula 7. (Karlin, S. and Altschul, S. F., Methods for assessing the statistical significance of molecular sequence features by using general scoring scheme, Proc. Natl. Acad. Sci. USA, Vol. 87, pp.2264-2268, 1990)

p value=1−exp[−K*N*M*exp(−λ*S)] (Formula 7)

Where,

N=the base length of the sequence A,

M=the base length of the sequence B,

λ=ln(q/p)

K==(q−p)2/q,

p=the probability of score 1 incurred, and

q=the probability of score −1 incurred.

This p value is a stochastic index. In many cases, for example, assuming that the homology between two compared DNA sequences is sufficiently high (a risk percentage of 10% or less) if the p value calculated using the blast algorithm for a candidate probe and other gene is equal to or less than 10%, the candidate probe can be discarded from the candidate probe list 13 to significantly reduce the potentiality of hybridization. On the other hand, if the base sequences are analogous to the overall regions of other gene sequences, there are many candidate probes, for which it is difficult to definitely determine whether they can be used as the probes only from an aspect of stochastic indexes with respect to the genes, such as CYP2C8, CYP2C9, CYP2C18, and CYP2C19, of cytochrome P450, which belong to the same super family. That is because the E value or the p value used in homology search may have different values depending on the length of the sequence even though the identity remains unchanged. For example, even if the p value calculated using the blast algorithm is 1.0, namely even if the homology between two compared sequences is probably almost zero (0), the identity may indicate any value of approx. 40% to approx. 75%. The objective of homology search can be attained by the assumption that if no match is observed between a DNA sequence and the gene sequences contained in the database (a null hypothesis), the null hypothesis gives a correct probability. The identity 75% means that 25% of sequences are not matched. Depending on the lengths of the sequences, two sequences being compared, between which 25% of sequences are not matched, may be different from one another at a high probability. For this reason, the p value is almost 1.0 and the homology between two sequences is determined to be zero (0). In fact, however, two sequences, between which the identity is 75%, hybridize one another at a hybridization temperature lower than approx. 30° C. Tm. The homology search algorithm gives no direct answer to the question about whether such hybridization occurs. To overcome this problem, it is desirable that in addition to the p value and the E value obtained from homology search, thermodynamic indexes, for example, melting temperature and free energy indexes, which more directly reflect the physiochemical reaction of probe-target hybridization, are used. In the step of the cross-hybridization filter 45, the candidate probes are discarded based on the their thermodynamic indexes, such as differences in melting temperature and in free energy.

Note that the sequence of the melting temperature filter 41, the secondary structural filter 42, the repetitive sequence filter 43, the analogous sequence filter 44, and the cross-hybridization filter 45 may not be limited to that shown in FIG. 4. For example, either of the secondary structural filter 42 or the repetitive sequence filter 43 may be used at the top of the step. It is desirable, however, that the analogous sequence filter 44, which usually takes a long time, is used in the latter part of the filtering step 14.

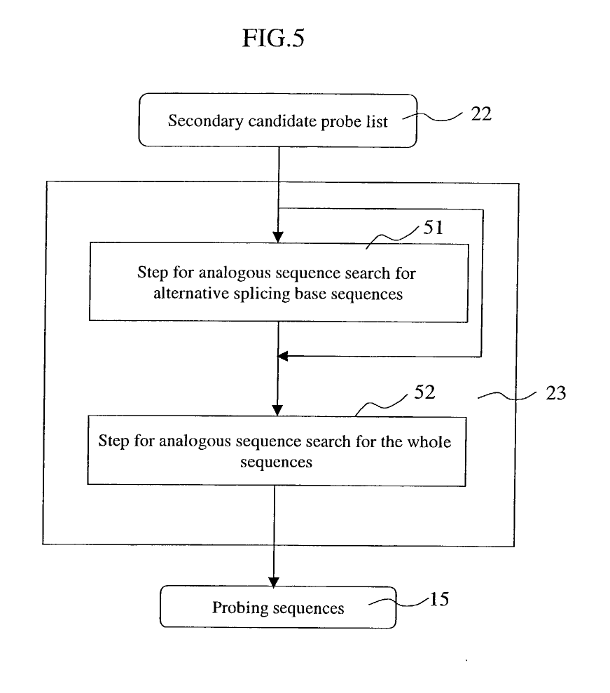

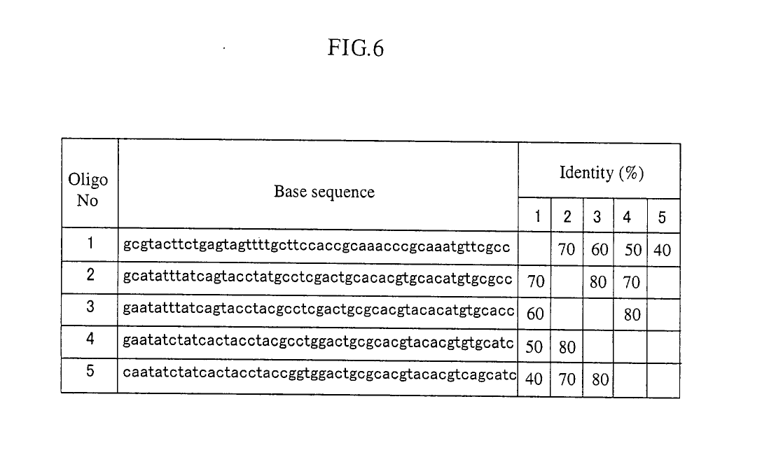

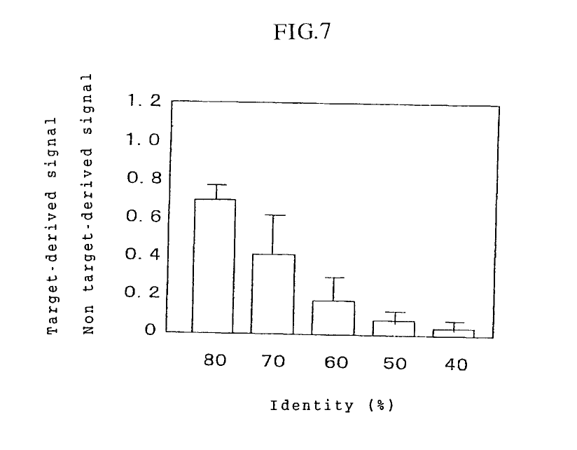

As shown in FIG. 5, the last re-examination step 23 also can be divided into more than one sub-step. The re-examination step 23, which is intended to supplement the analogous sequence filter step 44, comprises sub-steps, such as the sub-steps of analogous sequence search of alternative splicing base sequences 51 and analogous sequence search of all the base sequences 52. Alternative splicing is a phenomenon, in which more than one transcribed product (RNA) is produced from the same gene. Since more than one transcribed product is derived from the same gene, large parts of their sequences are often analogous. On the DNA chips, alternative-splicing base sequences may be measured separately, which requires a higher accuracy of search algorithm. To overcome this problem, it is desirable that the sub-step of analogous sequence search of alternative splicing base sequences 51 is used. Note that when it is not needed to measure alternative splicing base sequences separately, the sub-step 51 can be bypassed. In the sub-step of analogous sequence search of all the base sequences 52, all the base sequences registered in the databases including the base sequence databases, such as GENBANK, and the EST databases, such as dbEST, are searched for any analogous sequences. In addition to search within the same specie, analogous search may be executed across several species including human being, mouse, and rat. To examine the relationship between the identities and the potentialities of cross-hybridization, an experiment was conducted. Five synthesized DNAs (oligonucleotide DNAs or oligo DNAs) with a base length of 50 bp and different sequences were prepared for experimental use. These five oligo DNAs were used as probes. FIG. 6 shows the base sequences of the prepared oligo DNAs. In the right column of FIG. 6, the identities for a various combination of sequences are listed. In the experiment, individual oligonucleotides and one target DNA with the oligo DNA end fluorescent-labeled, which had a complementary sequence was selected among five kind of DNAs, applied the target DNA onto the DNA chip, which had oligo DNAs 1-5 fixed on the same base plate as shown in FIG. 6, and left as it was at a temperature of 62° C. for 12 hours to induce hybridization between the target DNA and the oligo DNAs. The intensities of the fluorescent signals derived from fluorescent labels were digitized using a fluorescent scanner. The experiment was repeated three times for each of five kinds of target DNAs and the results were summarized as shown in FIG. 7. In FIG. 7, a horizontal axis indicates the identities and a vertical axis indicates the quotients obtained by the division of the target-derived signals by the non target-derived signals. Error bars indicate standard deviations. The target-derived signal exhibits the intensity of the signal from hybridization between the target DNA and the probing DNA, between which the identity is 100%. For example, the intensity of the signal from hybridization between oligo 1 and the target having the sequence complementary to oligo 1 shown in FIG. 6 is a typical example. The non target-derived signal exhibits the intensity of the signal from hybridization between the target DNA with a identity, 80%, 70%, 60%, 50%, or 40% and the probing DNA except for the target-derived signal. Thus, at the point 1.0 along the vertical axis (target-derived/non target-derived signals) in FIG. 7, the intensities of the target-derived and non target-derived signals are identical. At that time, cross-hybridization (hybridization of which occurrence is not desirable) has occurred at the same level as that of intended target-probe hybridization, which disables the candidate probe to be used as a probe for the DNA chips because it does not measure individual gene expressions separately. According to FIG. 7, from an aspect of standard deviations, cross-hybridization between the sequences with the identities, 40%, 50%, or 60% is not significant, although cross-hybridization between an average of 40% of sequences with an identity of 70% and between an average of 60% of sequences with an identity of 80% was observed. The results summarized in FIG. 7 largely vary depending on the hybridization temperature and how to wash away the DNA array. Stringent washing of the DNA array reduces the potentiality of hybridization. For this reason, the results summarized in FIG. 7 are not applicable directly to all the DNA chips, although roughly speaking, a risk of occurrence of hybridization increases when the identity exceeds 80%.

As mentioned above, the motifs are the sites having the sequences relatively analogous to other gene sequences. Generally, the motifs are often defined by protein sequences. Since the protein sequence is produced by partitioning the DNA sequence by a unit of codon (a three-base length of sequence) and replacing the partitioned codons with amino acids, it is considered that the DNA sequences in addition to the protein sequences are also analogous in the motif regions. To prove this assumption, 1000 motif sequences were obtained from GENBANK and motif search was executed using PROSITE. The DNA sequences of the obtained protein motif sites were compared with the gene sequences having the same motifs. The results from comparison are shown in Table 1 to Table 3.

TABLE 1

| | | No. of | | | |
|---|---|---|---|---|---|
| | Motif base | appropriate | Identity | Identity | Identity |
| Motif name | length | genes | (minimum) | (average) | (maximum) |
| FORK_HEAD_1 | 41 | 2 | 100.0 | 100.0 | 100.0 |
| UBIQUITIN_1 | 77 | 9 | 90.9 | 95.7 | 100.0 |
| SPERACT_RECEPTOR | 113 | 8 | 90.3 | 93.1 | 100.0 |
| H5P70_1 | 23 | 8 | 65.2 | 81.8 | 100.0 |
| ADH_ZINC | 44 | 7 | 72.7 79 8 | 100.0 | |
| UDPGT | 131 | 12 | 64.1 | 77.9 | 100.0 |
| KRINGLE_1 | 17 | 11 | 52.9 | 76.3 | 100.0 |
| RNA_POL_IL_REPEAT | 20 | 43 | 40.0 | 68.0 | 100.0 |
| NUCLEAR_RECEPTOR | 80 | 31 | 51.3 | 66.7 | 100.0 |
| HSP70_3 | 44 | 11 | 45.5 | 65.5 | 100.0 |
| HSP70_2 | 41 | 10 | 39.0 | 65.5 | 100.0 |
| ATPASE_E1_E2 | 20 | 17 | 15.0 | 64.5 | 100.0 |
| ZINC_FINGER_C3HC4 | 29 | 9 | 37.9 | 62.0 | 100.0 |
| IG_MHC | 20 | 7 | 40.0 | 59.3 | 100.0 |
| CYTOCHROME_P450 | 29 | 32 | 27.6 | 54.6 | 100.0 |
| ABC_TRANSPORTER | 44 | 38 | 13.6 | 53.8 | 100.0 |
| ATP_GTP_A | 23 | 134 | 4.4 | 48.4 | 100.0 |
| INTERFERON_A_B_D | 56 | 10 | 23.2 | 73.4 | 96.4 |
| G_PROTEIN_RECEPTOR | 50 | 7 | 22.0 | 53.1 | 98.0 |
| ALDEHYDE_DEHYDR_GLU | 23 | 12 | 34.8 | 68.8 | 95.7 |
| PROTEIN_KINASE_TYR | 38 | 33 | 42.1 | 65.3 | 94.7 |
| ALDEHYDE_DEHYDR_CYS | 35 | 13 | 34.3 | 53.7 | 94.3 |
| SER_THRPHOSPHATASE | 17 | 3 | 70.6 | 78.4 | 94.1 |
| TRYPSIN_HIS | 17 | 6 | 52.9 | 74.1 | 94.1 |
| PRENYLATION | 11 | 29 | 20.0 | 44.7 | 90.9 |
| REL | 20 | 5 | 55.0 | 68.5 | 90.0 |
| RECEPTOR_CYTOKINES_2 | 20 | 16 | 40.0 | 66.6 | 90.0 |
| DAG_PE_BINDING_DOMAIN | 149 | 17 | 34.9 | 52.8 | 89.9 |
| DYNAMIN | 29 | 2 | 89.7 | 89.7 | 89.7 |
| WNT1 | 29 | 8 | 55.2 | 76.6 | 89.7 |
| PROTEIN_KINASE_ST | 38 | 60 | 10.5 | 57.4 | 89.5 |
| CASPASE_HIS | 44 | 10 | 18.2 | 47.8 | 88.6 |
| BH2 | 35 | 6 | 45.7 | 57.7 | 88.6 |
| IF | 26 | 3 | 65.4 | 74.4 | 88.5 |
| CYCLINS | 95 | 6 | 36.8 | 59.9 | 88.4 |
| CONNEXINS_1 | 41 | 10 | 65.9 | 74.8 | 87.8 |
| SMALL_CYTOKINES_CC | 125 | 11 | 40.8 | 56.6 | 87.2 |
| PI3_4_KINASE_1 | 44 | 10 | 47.7 | 66.1 | 86.4 |
| WD_REPEATS | 44 | 38 | 11.4 | 45.5 | 86.4 |
| HSF_DOMAIN | 74 | 3 | 75.7 | 80.6 | 85.1 |
| IGF_BINDING | 47 | 8 | 48.9 | 68.0 | 85.1 |

TABLE 2

| | Motif | No. of | | | |
|---|---|---|---|---|---|
| | base | appropriate | Identity | Identity | Identity |
| Motif name | length | genes | (minimum) | (average) | (maximum) |
| PDGF | 38 | 7 | 57.9 | 68.2 | 84.2 |
| HELIX_LOOP_HELIX | 47 | 17 | 14.9 | 40.8 | 83.0 |
| TRYPSIN_SER | 35 | 5 | 57.1 | 66.0 | 82.9 |
| CASPASE_CYS | 35 | 10 | 17.1 | 65.1 | 82.9 |
| INTEGRIN_ALPHA | 23 | 3 | 60.9 | 71.0 | 82.6 |
| CYTOCHROME_C | 17 | 13 | 35.3 | 55.4 | 82.4 |
| PI3_4_KINASE_2 | 62 | 9 | 43.6 | 66.2 | 82.3 |
| ZINC_FINGER_C2H2 | 62 | 24 | 11.3 | 38.7 | 82.3 |
| CONNEXINS_2 | 50 | 9 | 54.0 | 65.7 | 82.0 |
| HOMEOBOX_1 | 71 | 5 | 33.8 | 53.5 | 81.7 |
| HBGF_FGF | 71 | 13 | 38.0 | 53.0 | 81.7 |
| TGF_BETA | 47 | 3 | 74.5 | 76.6 | 80.9 |
| RECEPTOR_TYR_KIN_II | 26 | 3 | 65.4 | 74.4 | 80.8 |
| MYB_1 | 26 | 7 | 42.3 | 58.2 | 80.8 |
| RECEPTOR_TYR_KIN_III | 41 | 7 | 61.0 | 71.0 | 80.5 |
| DNA_MISMATCH_REPAIR_1 | 20 | 3 | 75.0 | 76.7 | 80.0 |
| IRF | 101 | 7 | 52.5 | 62.1 | 79.2 |
| POU_1 | 38 | 3 | 65.8 | 70.2 | 79.0 |
| TYR_PHOSPHATASE_1 | 38 | 7 | 44.7 | 62.8 | 79.0 |
| BHI | 56 | 4 | 51.8 | 61.9 | 78.6 |
| POU_2 | 41 | 3 | 75.8 | 76.4 | 78.1 |
| ETS_DOMAIN_1 | 26 | 3 | 61.5 | 69.2 | 76.9 |
| THYROGLOBULIN_1 | 101 | 3 | 63.4 | 67.7 | 76.2 |
| SIGMA54_INTERACT_1 | 41 | 3 | 63.4 | 68.3 | 75.6 |
| AAA | 56 | 9 | 8.9 | 41.0 | 75.0 |
| MYB_2 | 71 | 6 | 35.2 | 50.1 | 74.7 |
| CRYSTALLIN_BETAGAMMA | 47 | 11 | 14.9 | 40.2 | 74.5 |
| C2_DOMAIN_1 | 47 | 3 | 14.9 | 35.5 | 74.5 |
| EGF_1 | 35 | 17 | 31.4 | 50.8 | 74.3 |
| HMA | 89 | 12 | 36.0 | 54.8 | 74.2 |
| ATPASE_ALPHA_BETA | 29 | 4 | 41.4 | 54.6 | 72.4 |
| C_TYPE_LECTIN_1 | 83 | 3 | 65.1 | 68.7 | 72.3 |
| NNMT_PNMT_TEMT | 50 . | 2 | 72.0 | 72.0 | 72.0 |
| UCH_1 | 50 | 2 | 720 | 72.0 | 72.0 |
| CARBOXYLESTERASE_B_2 | 32 | 2 | 71.9 | 71.9 | 71 9 |
| DNAJ_1 | 59 | 5 | 54.2 | 64.9 | 71.2 |
| GRAM_POS_ANCHORING | 17 | 3 | 52.9 | 60.8 | 70.6 |
| INSULIN | 44 | 2 | 70.5 | 70.5 | 70.5 |
| VWFC | 140 | 2 | 70.0 | 70.0 | 700 |
| TNF_1 | 50 | 8 | 38.0 | 53.9 | 70.0 |

TABLE 3

| | | No. of | | | |
|---|---|---|---|---|---|
| | Motif base | appropriate | Identity | Identity | Identity |
| Motif name | length | genes | (minimum) | (average) | (maximum) |
| HIT | 56 | 2 | 69.6 | 69.6 | 69.6 |
| ATPASE_NA_K_BETA_1 | 62 | 4 | 56.5 | 61.0 | 69.4 |
| EGF_2 | 35 | 9 | 40.0 | 53.6 | 68.6 |
| RECEPTOR_CYTOKINES_1 | 38 | 10 | 34.2 | 48.6 | 68.4 |
| BH3 | 44 | 6 | 38.6 | 50.5 | 68.2 |
| DNA_MISMATCH_REPAIR_2 | 50 | 3 | 66.0 | 67.3 | 68.0 |
| ASX_HYDROXYL | 35 | 3 | 60.0 | 62.9 | 65.7 |
| INTERLEUKIN_1 | 62 | 3 | 38.7 | 48.4 | 64.5 |
| CARBOXYLESTERASE_B_1 | 47 | 2 | 63.8 | 63.8 | 63.8 |
| ETS_DOMAIN_2 | 47 | 3 | 53.2 | 58.9 | 63.8 |
| ATPASE_NA_K_BETA_2 | 47 | 4 | 53.2 | 58.2 | 63.8 |
| ER_TARGET | 11 | 2 | 63.6 | 63.6 | 63.6 |
| NGF | 41 | 2 | 63.4 | 63.4 | 63.4 |
| BH4_1 | 62 | 3 | 58.1 | 61.3 | 62.9 |
| CARBOXYPEPT_ZN_2 | 32 | 2 | 62.5 | 62.5 | 62.5 |
| DNA_POLYMERASE_B | 26 | 2 | 61.5 | 61.5 | 61.5 |
| DNA_POLUMERASE_X | 59 | 3 | 57.6 | 58.8 | 61.0 |
| INTEGRIN_BETA | 41 | 5 | 46.3 | 52.7 | 61.0 |
| RNA_POL_D_30KD | 122 | 2 | 59.8 | 59.8 | 59.8 |
| RNAPOL_L_13KD | 86 | 2 | 58.1 | 58.1 | 58.1 |
| RAS_GTPASE_ACTIV_1 | 44 | 2 | 56.8 | 56.8 | 56.8 |
| RNP_1 | 23 | 5 | 26.1 | 43.5 | 56.5 |
| EF_HAND | 38 | 8 | 26.3 | 39.3 | 55.3 |
| DEATH_ATP_HELICASE | 29 | 2 | 55.2 | 55.2 | 55.2 |
| INTERLEUKIN_7_9 | 29 | 2 | 55.2 | 55.2 | 55.2 |
| GATA_ZN_FINGER | 74 | 2 | 54.1 | 54.1 | 54.1 |
| CTCK_1 | 113 | 2 | 52.2 | 52.2 | 52.2 |
| TFIIS | 113 | 2 | 52.2 | 52.2 | 52.2 |
| ZINC_PROTEASE | 29 | 2 | 51.7 | 51.7 | 51.7 |
| LIPASE_SER | 29 | 3 | 41.4 | 44.8 | 48.3 |
| MITOCH_CARRIER | 29 | 2 | 44.8 | 44.8 | 44.8 |
| SUGAR_TRANSPORT_2 | 77 | 3 | 26.0 | 33.8 | 44.2 |
| ZP_DOMAIN | 122 | 2 | 43.4 | 43.4 | 43.4 |
| EPO_TPO | 83 | 2 | 42.2 | 42.2 | 42.2 |
| ADK_SHORT | 86 | 2 | 41.9 | 41.9 | 41.9 |
| INTERLEUKIN_6 | 77 | 2 | 41.6 | 41.6 | 41.6 |
| THIOL_PROTEASE_HIS | 32 | 2 | 40.6 | 40.6 | 40.6 |
| SUBTILASE_ASP | 32 | 2 | 31.3 | 31.3 | 31.3 |
| DNA_POLYMERASE_A | 59 | 2 | 27.1 | 27.1 | 27.1 |
| HTH_ARAC_FAMILY_1 | 131 | 2 | 26.0 | 26.0 | 26.0 |

In this table, the columns show motif names, motif base lengths, the number of genes having the appropriate motif among 1000 protein sequences mentioned above (No. of appropriate genes), minimum identities between two sequences randomly selected from appropriate genes (identity (minimum), identities averaged over all the appropriate genes (identity (average), and maximum identities, starting from the leftmost column. Table 1, Table 2, and Table 3 list the motifs in the descending order of identities.

As known from Table 1, Table2, and Table 3, sequence analogy is high not only between protein sequences but also between DNA sequences in the motif sites. In one third or more of motif sites, the identity is higher than 70% and in about half of motifs, the identity is higher than 60%. From the results shown in FIG. 6, FIG. 7, Table 1, Table 2, and Table 3, it is known that the use of the motif sites as the probes may cause significant hybridization to occur at a rate of about one third. Accordingly, it is desirable that no motif sites are used as the probes with an exception of the DNA chip, on which only motif sites are intentionally fixed. Intentionally using the motif sites as the probes, the target DNA fragments of more than one known and unknown genes having said motifs can be hybridized. New genes having said motifs can be cloned by peeling the target DNAs, which have been hybridized, off from the probes by any of the methods, such as ultrasound and thermal treatments, and determining the base sequences of the target DNAs. Since the genes having the same motifs are likely to have similar functions, it is relatively easy to forecast the functions of the new genes from the functions of the known genes having the same motifs as those of the new genes. Thus, on the DNA chip, which is intended to clone the new genes having similar functions, the probes including the partial or whole motif sites may be used. On the other hand, to prepare the DNA chip for measuring the expression distributions of known genes and ESTs, it is a requirement useful in preventing cross-hybridization from occurring that no motif sites are included in the DNA chip. Further, to reduce the time required for probe design, it is useful that the motif sites are masked in the masking step 31 shown in FIG. 3 prior to the preparation of the DNA chip so as not to include the motif sites. The method for reducing the time required for probe design by masking the motif sites will be further described in EXAMPLES.

One of characteristics of the prevent invention is in that the sequence sites analogous among more than one gene are masked in the filtering step prior to the preparation of the DNA chip to improve the speed of computation. In the prevent invention, for example, a region including the motif site is masked. That is because even for species, such as human being, mouse, and rat, of which all the DNA sequences have not necessarily determined, the motif sites analogous among more than one gene can be forecasted at a high probability. Note that for the species, such as E. coli bacteria and yeast, of which all the sequences have been determined, the identities can be calculated using any of homology search algorithms, for example, BLAST, to identify analogous sequence sites. In the present invention, the filtering step is executed using either the motifs or the homology search algorithm.

It is very dangerous to assess the potentiality of cross-hybridization using the identities only. That is because it cannot be identified whether the distribution of any mismatch between two sequences being compared occupies uniformly the whole sequence or locally the partial sequence. For example, for a sequence (a sequence, of which bases bind complementarily to others) of which 40 base pairs are completely matched and the remaining 40 base pairs are completely mismatched, the identity of a 80 base-pair sequence is 50%. While, the identity of a 80 base-pair sequence, in which match and mismatch are distributed alternately for individual bases, is also 50%. Although this is an extreme example, the potentiality of cross-hybridization is definitely higher in the former case than in the latter case even though they have the same identity of 50%.

Since the identities (%) and the statistically significant level values (p values or E values) calculated using the homology search method such as BLAST have different meanings, the p values may differ from one another even though the identities are the same. On the contrary, the identities may differ from one another even though the p values are the same. This means that the results from the assessment may be different depending on which are used in assessing the potentialities of cross-hybridization, identities or statistically significant level values. This ambiguity can be eliminated using physiochemical indexes Tm difference and ΔG difference.

Another one of the characteristics of the present invention is in that the potentialities of probe cross-hybridization are determined based on the physiochemical indexes, such as melting temperature difference and free energy difference. For example, using the homology search algorithm such as BLAST, the potentialities of cross-hybridization are determined based only on the enumeration of alphabets ATGC. In contrast, the indexes according to the prevent invention allow the potentialities of cross-hybridization to be determined from a far solid aspect of phenomenon.

Examples of identities, statistically significant level values, Tm differences, and ΔG differences obtained actually in the probe design process are shown in Table 5. In Table 5, the column of probe-designed genes lists the Unigene symbol names of the genes, for which probes were designed, and the column of most-analogous other genes lists the Unigene symbol names of other genes, which have the sequences most analogous to the sequences of the probe-designed genes. In the columns of the right side, the probe sequences designed for the probe-designed genes, the identities between the probing sequences and the gene fragment sequences of other genes having the highest homology to the probing sequences, statistically significant level values, Tm differences, and ΔG differences are listed.

The case 1, the case 2, and the case 3 in Table 1 show the examples of the sequences having the same identities and the different statistically significant level values. The case 4, the case 5, the case 6, and the case 7 in Table 1 show the examples of sequences having the same statistically significant level values and the different identities. Using the indexes of Tm differences and ΔG differences, with respect to all the cases from the case 1 to the case 7, the descending order of potentialities of cross-hybridization can be correctly assessed.

TABLE 4

| | | No. of times |
|---|---|---|
| | Time require for | analogous sequence |
| | probe design | filter is applied |
| Calculated time | 3.1 days | 1.3 |
| with motif | | |
| information input | | |
| step | | |
| Calculated time | 9.4 days | 3.8 |
| without motif | | |
| information input | | |
| step | | |

### EXAMPLES

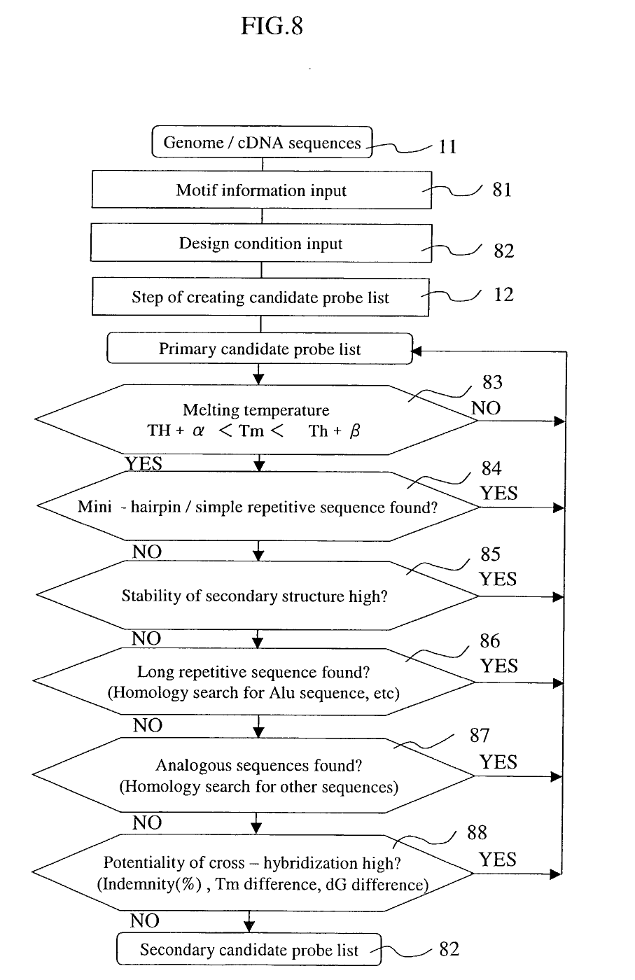

An example of flowcharts of software for designing the probes for DNA probe arrays, which was built according to the method disclosed in DETAILED DESCRIPTION OF THE INVENTION, is shown in FIG. 8. The flowchart shown in FIG. 8 explains a workflow until the creation of the secondary candidate probe list 22 shown in FIG. 2. In FIG. 8, in addition to the steps shown in FIG. 2, the motif information input sub-step 81 to mask the motif sites is included in the list-up step 12 and the design condition input sub-step 82, in which the conditions required for calculating melting temperatures, repetitive sequences, and secondary structures are entered, is included in the filtering step 14. It can be said, however, that FIG. 2 and FIG. 8 are the same because the sub-steps 81 and 82 are simply used to enter the information required in the steps 12 and 14 in FIG. 2. Note that the melting temperature filter 83 shown in FIG. 8 corresponds to the melting temperature filter 41 shown in FIG. 4 though the shapes of their boxes are different. In FIG. 8, Th indicates the hybridization temperature, Th+α indicates the lower limit of the preset melting temperature, and Th+β indicates the upper limit of the preset melting temperature. α and β may have either positive values or negative values. The secondary structural filter 85 shown in FIG. 8 corresponds to the secondary structural filter 42 shown in FIG. 4, the analogous sequence filter 87 shown in FIG. 8 to the analogous sequence filter 44 shown in FIG. 4, and the cross-hybridization filter 88 shown in FIG. 8 to the cross-hybridization filter 45 shown in FIG. 4, respectively. Note that the repetitive sequence filter 43 shown in FIG. 4 is divided into two sub-steps, the mini-hairpin repetitive sequence filter 84 and the long repetitive sequence filter 86 in FIG. 8 for improving the speed of computation. The sequence of the sub-steps shown in FIG. 8 was determined considering that it is useful in computation to execute the sub-steps 84 and 85 using different algorithms instead that the sub-steps 84 and 86 are executed using the same algorithm and it is preferable in improving the speed of computation that the sub-step 86 requiring less time than the sub-step 85 is put at the point before the sub-step 85 and the sub-step 86 requiring more time than the sub-step 85 is put at the point after the sub-step 85. Note that the sequence of sub-steps 83 to 88 may be determined so as to minimize the time required for computation through the filtering step 14 and it is not necessary to follow the sequences of sub-steps shown in FIG. 4 and FIG. 8. Now, the steps are described in detail following the sequence shown in the flowchart drawn in FIG. 8.

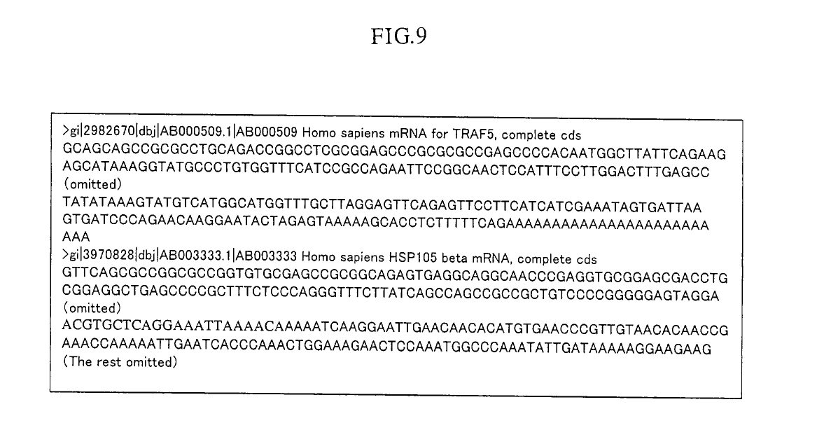

As shown in, for example, FIG. 9, a genome/cDNA sequence 11 is entered into a probe design system by reading a text file containing data described in any of formats such as FASTA. FIG. 9 shows the top part of the file containing approx. 40,000 human cDNA sequences. Note that one or more compressed or uncompressed files may be used for containing information on the genome/cDNA sequence 11. Moreover, any files in different file formats, GenBank, EMBL, and DDBJ in addition to FASTA, may be used. The files need not always to contain base sequences as shown in FIG. 9 and only ID numbers and the like, which allow the base sequences to be referenced, are required, such as GenBank action numbers, Unigene ID numbers, and GI numbers.

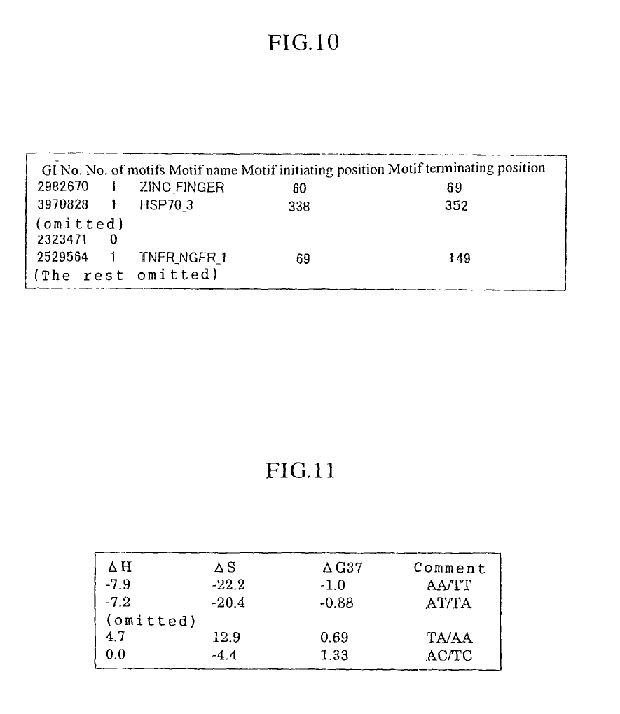

In the motif information input sub-step 81, the text file containing motif information in the format shown in, for example, FIG. 10 is read. In FIG. 10, the GI numbers of cDNA sequences, No. of motifs contained in the cDNA sequence, motif names, the initiating base position of a motif (number), and the terminating base position of a motif (number) are listed, starting from the leftmost. In the rows in FIG. 10, the individual sequences shown in FIG. 9 are listed. Based on the motif initiating positions and terminating positions shown in FIG. 10, the regions corresponding to the motif among the base sequences contained in the file shown in FIG. 9. Even though the file shown in FIG. 10 has not been prepared, based on the GI numbers and the like described in the file containing information on the genome/cDNA sequence 11, the DNA sequence and protein sequence motif information can be obtained by searching any of databases such as GenBank. This means that the file shown in FIG. 10 is not always needed assuming that DNA or protein sequences are obtained from the database using the action numbers and the like of GenBank shown in FIG. 9 and the motif sites are obtained from the obtained DNA or protein sequences through computation.

The effect of pre-masking of the motif sites on the reduced computation time in designing 5000 DNA probes is shown in Table 4.

TABLE 5
Examples of Identities, statistically significant
level values, Tm differences, and ΔG differences

| | | | | Statistically | Tm | ΔG |
|---|---|---|---|---|---|---|
| | Probe-designed | Most-analogous | | significant | difference | difference |
| Case | gene | other gene | Identity (%) | level values | (° C.) | (Kcal/mol) |
| 1 | GSTM1 | GSTM2 | 76.0 | 1.17E+01 | 20.1 | −39.5 |
| 2 | GJB2 | GJB6 | 76.0 | 4.40E+01 | 22.6 | −42.3 |
| 3 | ABCD1 | ABCD2 | 76.0 | 9.87E+01 | 23.3 | −45.2 |
| 4 | RAC1 | RAC2 | 75.6 | 1.00E+01 | 28.9 | −48.0 |
| 5 | GNB3 | GNB2 | 73.1 | 1.00E+01 | 24.4 | −46.3 |
| 6 | UGT1A9 | UGT2B | 70.9 | 1.00E+01 | 24.9 | −47.3 |
| 7 | CDK6 | CDK4 | 69.4 | 1.00E+01 | 35.6 | −49.8 |

By masking the motif sites, the time require for computation was reduced to approx one third. That is because by pre-masking the analogous sites, called motif sites, of more than one gene, the chance, at which the candidate probes passed the analogous sequence filter 87 of the later filtering step 14 at the fist time, increased. Time-consuming computation is of no use if many candidate probes are discarded in the filtering step 14, while pre-masking the motif sites is useful for eliminating the need for this time-consuming computation.

The conditions for design to be entered in the design condition input sub-step 82 include, for example, probing base lengths, salt concentrations of hybridization solutions, hybridization temperatures, the upper limits, upper limits, and target values of probe melting temperatures. The parameters to be used for calculating the probe melting temperatures and the stabilities of secondary structures can be read from the file, for example, shown in FIG. 11. FIG. 11 shows the thermodynamic parameters to be used in calculating the melting temperatures using the Nearest Neighbor (NN) method (SantaLucia, J, Jr. A unified view of polymer, dumbbell, and oligonucleotide DNA nearest-neighbor thermodynamics, Porc. Natl, Acad. Sci. USA, vol.95, p. 1460-1465, 1998).

In FIG. 11, the columns list ΔH, ΔS, and ΔG (37° C.). As known from the explanation in the comment column, the first row describes ΔH, ΔS, and ΔG (37° C.) of the two-base sequence, AA/TT. The relationship among the melting temperature (Tm), ΔH, and ΔS can be represented by the following formula 8.

Tm=ΔH/(ΔS+R*ln(Ct)) (Formula 8)

Where,

R=a gas constant, and

Ct=a concentration of oligonucleotide.

An example of the computation of ΔG using the NN method is shown in formula 9.

For example, if the base sequences are as shown below,

5′ C-G-T-T-G-A 3′

3′ G-C-A-A-C-T 5′free energy ΔG (NN) calculated by the NN method

ΔG(NN)=ΔG(CG/GC)+Δ(GT/CA)+ΔG(TT/AA)

$$+ Δ G ( TG / AC ) + ΔG ( GA / CT ) + Δ G ( init ) \\ \begin{matrix} = - 2.17 - 1.44 - 1.00 - 1.45 - 1.30 + 0.98 + 1.03 \\ = - 5.35 k cal / mol\end{matrix} \qquad ( Formula 9 )$$

In the papers, individual values are summarized in numerical tables. ΔS and ΔH can be also calculated using the formula 9.

Furthermore, by correcting the solution with the salt concentration using for example the formula 10, the stability of the complementary bond in the solution (free energy) ΔG (in the solution) can be calculated.

ΔG(in the solution)=ΔG(NN)−0.175*ln[Na+]−0.20 (Formula 10)

Where,

[Na+]=a salt concentration

Note that two types of methods, the NN method and the empirical equation-based method, may be used in calculating the Tm of the DNA sequence (Wetmure, J. G., DNA probes: Applications of the principles of nucleic acid hybridization, Criti. Rev in Biochem. And Mol. Biol., vol.26, p.227-259, 1191). Empirical equation-based Tm calculations can be represented by the formula 11 for DNA-DNA hybridization, by the formula 12 for DNA-RNA hybridization, and by the formula 13 for RNA-RNA hybridization.

Tm=81.5+16.6*log([Na⁺]/(1+0.7[Na⁺]))+0.41*(%GC)−500/length (Formula 11)

Where,

[Na+]=a salt concentration,

%GC=a GC ratio, and

length=a base length.

Tm=67+16.6*log([Na^{+]/(}1+0.7[Na⁺]))+0.8*(%GC)−500/length (Formula 12)

Where,

[Na⁺]=a salt concentration,

%GC=a GC ratio, and

length=a base length.

Tm=78+16.6*log([Na⁺]/(1+0.7[Na⁺]))+0.7*(%GC)−500/length (Formula 13)

Where,

[Na⁺]=a salt concentration,

%GC=a GC ratio, and

length=a base length.

The NN method used in the formulae 8, 9, and 10 is well suited for the DNA sequences having less than 50 base pairs, while the empirical equation-based method used in the formulae 11, 12, and 13 is well suited for the DNA sequences having 50 or more base pairs. According to the above-mentioned paper written by J. Santa Lucia Jr., it is suggested that the NN method has been improved so that calculations can be made without depending on the base lengths. Thus, either the NN method or the empirical equation-based method may be used depending on the length of the sequence, alternately, for example, an average of the values obtained from the results of the calculations by the two methods may be used in calculating the Tm of the DNA sequence.

The motif sites obtained in the motif information input sub-step 81 are masked in the masking step 31 shown in FIG. 3. Next, depending on the lengths of the base sequences entered in the design condition input sub-step 82, the probe cutting sub-step 32 shown in FIG. 3 is executed to create the primary candidate probe list 21.

In the next sub-step of melting temperature filter 83, the melting temperatures (Tm) for individual candidate probes registered in the primary candidate probe list 21 are calculated and the calculated Tms are compared with the hybridization temperatures (Th) to discard the candidate probes, which deviated from a given range, for example, any DNA sequences deviated from Th+α by Th+β (α and β may have either positive or negative values), from the primary candidate probe list 21. Next, the differences between the melting temperatures of the candidate probes remaining in the primary candidate list 21 and the target values for the probe melting temperatures can be calculated to sort so that the candidate probes having the melting temperatures approximate to the target values for the probe melting temperatures occupy the upper positions in the list with the most approximate one at the top of the list. This sort process is not always needed but useful in designing the probes having the melting temperatures more approximate to the target values for the probe melting temperatures in a shorter period.

The next sub-step of mini-hairpin/simple repetitive sequence filter 84 is described. Generally, single-strand DNAs have several complementary base pairs formed with its part bent in the solution. This bent structure is called a hairpin. It has been known that the hairpin structures having abnormally higher thermo-stabilities, such as the sequence 1 and the sequence 2, are observed among these hairpin structures (Hirao and Miura, Special Structure of Single-Strand DNA-Mini-Hairpin Structures Having Abnormally Higher Thermo-Stabilities, Protein/Nucleic Acid/Enzyme, vol. 40, p. 1583-1592, 1995). The Tm of the sequence 1 reaches 76° C. The temperature 76° C. is significantly higher than those of common hybridization temperatures (a range of 40-62° C.). For this reason, when the sequence 1 is included in the fixed DNA fragment, the probability that no complementary bond is formed in the sequence sites or the wider regions is far high in hybridization. It can be said in some sense that in the state where no complementary bond is formed, mismatch may occur between the sequences. As mentioned above, any mismatch between two DNAs drops the Tms of the DNAs, leading to reduction in hybridization accuracy. For this reason, it is desirable that mini-hairpin structures, for example, the sequence 1 and the sequence 2, are not included in the fixed DNA fragments as possible. The sequence, which may deteriorate the hybridization accuracy, except for the sequence 1 and the sequence 2, includes a simple repetitive sequence. Examples of simple repetitive sequences are two-base repeats and three-base repeats. The two-base repeat is the sequence, in which, for example, two bases,

AT[AT]ₙAT(n=0, 1, 2 . . . ) (Sequence 3)

GC[GC]ₙGC(n=0, 1, 2 . . . ) (Sequence 4)

are repeated many times. The three-base repeat is the sequence, for example, three bases,

CXG[CXG]ₙCXG(note that X=any of A, T, G, and C, n=0, 1, 2 . . . ) (Sequence 5)

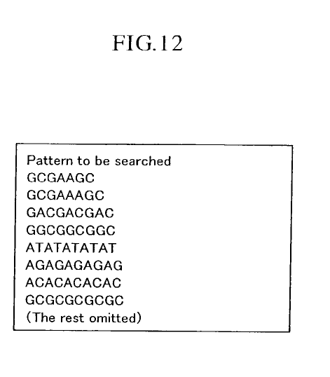

are repeated many times. In the sub-step 84, using, for example, the string pattern matching algorithm, two-base or three-base repeats are searched to discard the candidate probes having the sequences, which may deteriorate the hybridization accuracy, from the primary candidate probe list. Pattern matching may be applied to the mini-hairpins, the complementary sequence of repeat sequences (CGCTTTCG for GCGAAAGC), the reversed sequences (CGAAAGCG for GCGAAAGC), and the complementary reversed sequences (GCTTTCGC for GCGAAAGC). To improve the speed of computation, the strings may be converted into the numeric characters to compare with the sequences through shift computation. In the sub-step 84, pattern matching may be applied to the sequences by reading the file containing the pattern sequences (the patterns to be searched), to which pattern matching is applied, shown in, for example, in FIG. 12. In FIG. 12, more than one pattern to be searched, which are partitioned with a line feed (LF), are stored.

The next sub-step of secondary structural filter 86 is described below. It has been known that single-strand DNAs have various types of higher-order structures in addition to the hairpin structure by means of hydrogen bond between the bases and these higher-order structures including the hairpin structure are referred to as secondary structures together. If the stability of the secondary structure cannot be negligible as a result of the comparison with the stability of probe-target hybridization, the candidate probe is discarded from the primary candidate probe list 21. The secondary structures include, for example, the hairpin loop structure, internal loop structure, and the bulge loop structure. It can be determined whether the forecasted secondary structure is significantly stable by comparing the ΔG of the secondary structure obtained by the thermodynamic calculation as shown in the formula 1 and the ΔG of probe-target hybridization.

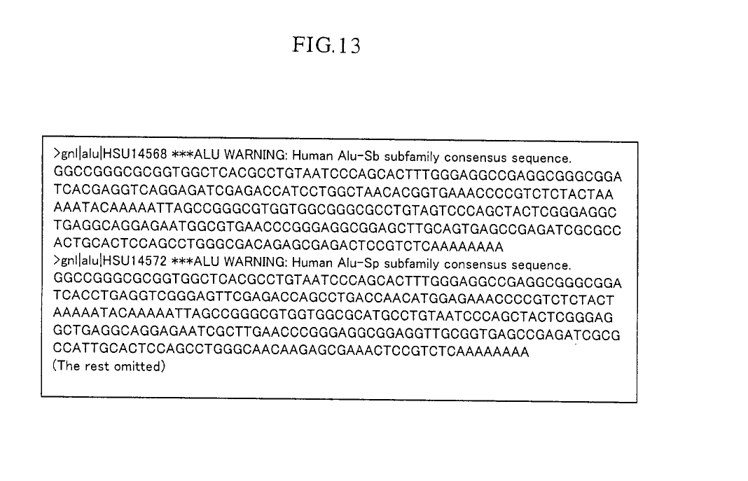

The next sub-step of long repetitive sequence filter 86 is described below. it has been known that a human DNA includes the sequences having many repetitive sequences specific to human being, called Alu sequences. Many Alu sequences are included in the human DNAs. Furthermore, the Alu sequences are included in the mRNAs, in particular, a 5′ non-translation region and a 3′ non-translation region. Assuming that a DNA molecule has been mixed in the mRNA sample to be measured, the mixed DNA molecule may be also fluorescent labeled because of the presence of the Alu sequence part in the fluorescent labeling step using the mRNA as a template. Furthermore, it may be considered that the labeled DNA molecule including the Alu sequence part is complementarily bonded with the fixed DNA fragment. This suggests that not only the mRNA, which is intended to measured, but also the DNA may be measured. To avoid a mistake in analysis due to the mixed DNA, it is desirable that no sequence part having significantly high homology to the Alu sequence is included in the fixed DNA fragments. Since the Alu sequence includes many repetitive structures, it may have a high-order structure, which is likely to cause mismatch in the same manner as that for the mini-hairpin structure. Also from this aspect, it is desirable that no sequence part having significantly high homology to the Alu sequence is included in the fixed DNA fragments. For example, by increasing the homology between the Alu sequence and the fixed DNA fragment by 10% compared with the statistically significant level value (p value) calculated using the BLAST algorithm, the homology between the sequences may be significantly reduced. The BLAST algorithm described in the paper written by Altschul, S. F. et al. (Basic local alignment search tool, J. Mol. Biol., Vol. 215, p. 403-410, 1990) may be used. Depending on the results of search for homology to the Alu sequence, the candidate probes having high homology to the Alu sequence are described from the primary candidate probe list 21. For human being, it has been known that other repetitive sequences are included than the Alu sequence, such as LINE (Long Interspersed Elements), SINE (Short Interspersed Elements), LTR retro-transposon, and DNA tansposon. For the species other than human being, it has been known that several other repetitive sequences corresponding to the Alu sequence are included. The homology to these repetitive sequences may be searched in the same manner as that for the Alu sequence to discard the candidate probes having high homology to them from the primary candidate probe list 21. To obtain the sequences to be used in homology search, the file containing the repetitive sequences, such as the Alu sequence, in the FASTA format as shown in FIG. 13 may be read.

In the next sub-step of analogous sequence filter 87, homology search is executed between the probing sequences fixed on a single DNA probe array or the gene sequences (genome sequence, cDNA sequence) including probing sequences and the candidate probes. Among the candidate probes, the candidate probes having high homology to other probing sequences or the gene sequences are discarded from the primary candidate probe list 21 because they cross-hybridize with the fixed DNA fragments. In sub-step 87, homology to all the gene sequences thought to be approx. 40,000 for human being, all the transcribed products thought to be approx. 100,000 for human being, or the total human genome sequence may be calculated, for example with respect to the specie of human being. The samples to be measured include many different genes other than the fixed DNA fragments and the intended genes to be hybridized. If these different genes are hybridized, they are also measured even though the measurement of only the genes to be hybridized with the individual fixed DNA fragments have been intended. To overcome this problem, it is desirable that the candidate DNA fragment sequences fixed on the DNA probe array is compared with the DNA sequences of a group of genes, which may be included in the sample to be measured so that no DNA sequences having significantly high homology are selected for the fixed DNA fragments. All the known sequences of the species, such as human being, rodentia, yeast, and E. coli., are contained in the gene sequence databases including GenBank. Assuming that DNA sequence homology between the fixed DNA fragments and the gene sequences, for example, of human being obtained from the databases including GenBank exceeds the statistically significant level value (p value) calculated using the BLAST algorithm by 10%, higher accuracy of measurement can be executed. As shown in FIG. 2, in the sub-step of analogous sequence filter 87, time-consuming homology search to other genes may be executed using the high-speed algorithm (a high-speed algorithm with less accuracy, such as the BLAST algorithm) and in the re-examination step 23 shown in FIG. 5, homology search may be executed using the low-speed algorithm (a low-speed algorithm with high accuracy, such as the Smith-Waterman algorithm).

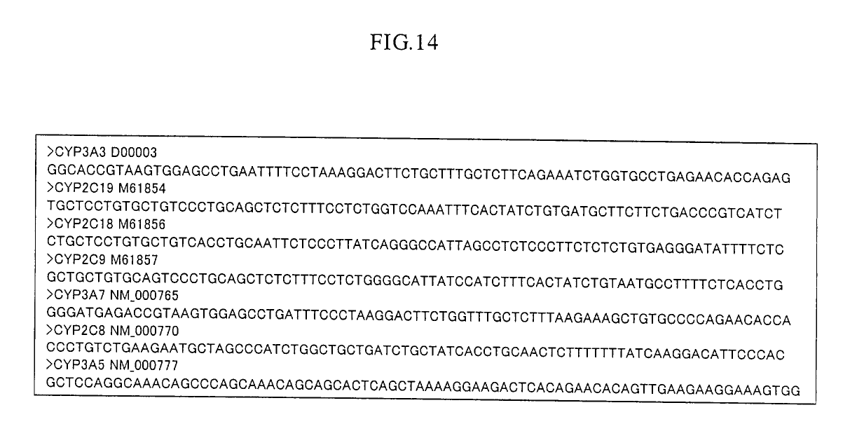

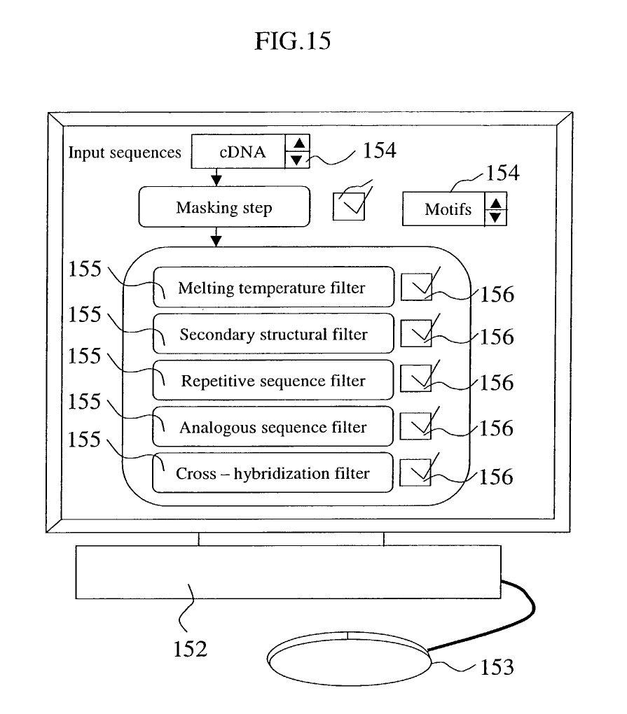

The last sub-step of the filtering step, the cross-hybridization filter 88 is described below. As mentioned above, a physiochemical process, probe-target hybridization, has not been necessarily considered in homology search. For this reason, the potentiality of cross-hybridization is checked for one or more candidate probes, which passed the sub-step of analogous sequence filter 87. For example, the identities between the candidate probes and other gene sequences having the highest homology, Melting temperature differences (Tm differences), and free energy differences (ΔG differences) are calculated. The candidate probes having significantly high identities between them and other gene sequences are discarded from the primary candidate probe list 21. The significantly high identities exceed 80%, for example, as shown in FIG. 7. The candidate probes having significantly small Tm differences from other genes are discarded from the primary candidate probe list 21. The significantly small Tm differences are, for example, below 15° C. In addition, the candidate probes having significantly small ΔG differences from other genes are discarded from the primary candidate probe list 21. It may be considered that by assessing the potentiality of cross-hybridization based on the physiochemical indexes, the potentiality of cross-hybridization for the prepared DNA probe array can be reduced to almost zero (0). The secondary candidate probes designed in a series of sub-steps shown in FIG. 8 are registered in the secondary candidate probe list 22 as shown in FIG. 14. Next, the list is subjected to the re-examination step 23 and others and finally the probing sequence 15 is determined.

FIG. 15 shows an example of interfaces according to the prevent invention. Interface hardware comprises, for example, a display device 151, a CPU 154, and an input device 153. The display device 151 provides a group of buttons including select buttons 154, display buttons 155, and check buttons 156, allowing the user of the present invention to enter the desired conditions by clicking them or taking any other actions at the input device 153. The select buttons 154 can be used to view all the selective choices to the user and select the desired one. For example, it may be selected whether the genome DNA or cDNA sequence is entered, or whether the motifs are masked or not. Clicking the display buttons 155 enables the user to view the input screen with detailed information or the help screen. The check buttons 156 are toggle buttons and the step associated with the button checked in is executed, while the step associated with the button checked out is omitted. Based on the conditions entered by the user, the list-up step 12, the filtering step 14, and the re-examination step 23 of the present invention can be combined or the sequence of the steps can be automatically optimized for software processing. Thus, even the user, who does not well known of the internal mechanism of software, can execute the fastest software processing.

By implementing the prevent invention, the probing sequence having no potentiality of cross-hybridization, equal Tms, and less secondary structures can be achieved.

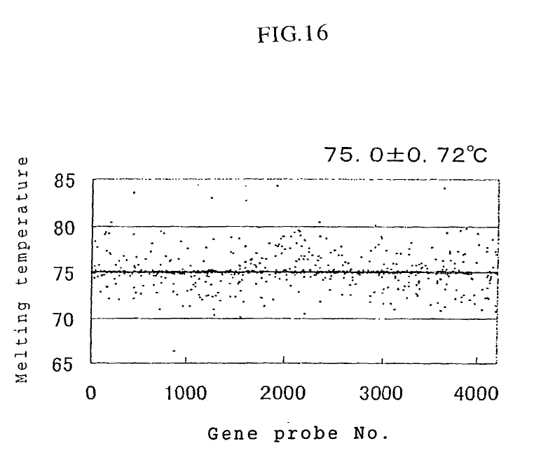

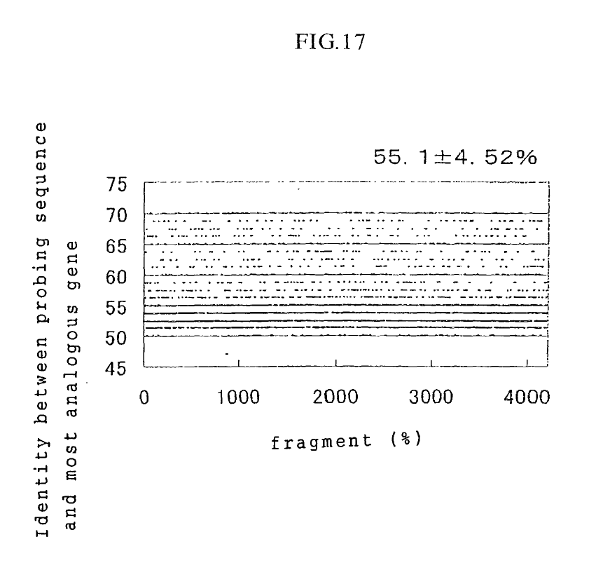

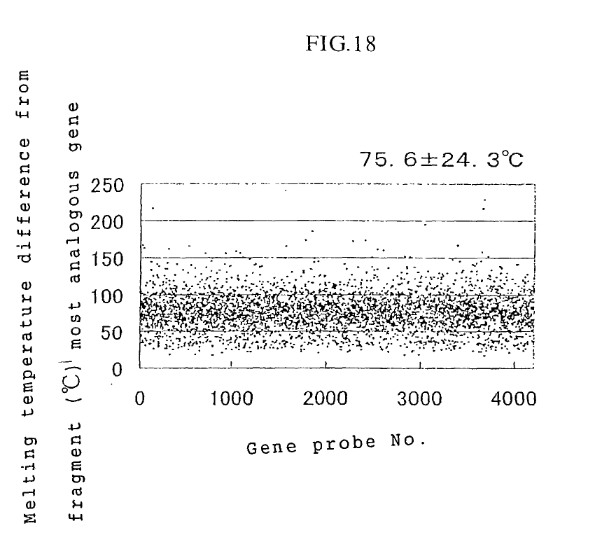

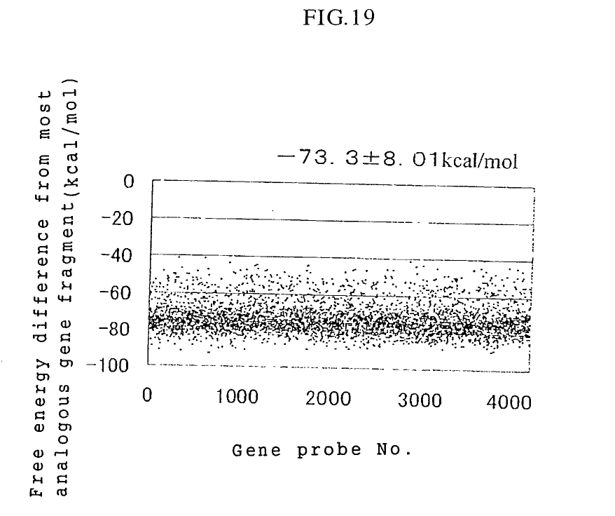

The oligonucleotide probes having 80 bases were designed for 4,274 known rat genes registered in Unigene. From the design process, the probing sequences could be determined for 4,216 genes (98.6%) out of 4,274 genes. The characteristics of 4,216 probing sequences are shown in FIG. 16 through FIG. 19. FIG. 16 shows the melting temperatures of 4,216 probes. In FIG. 16, probe numbers are indicated along a horizontal axis and the probe melting temperatures (° C.) are indicated along a vertical axis. A target value for the melting temperature was set to 75° C. in designing. The sequences having the melting temperatures most approximate to the target value, which satisfies all the conditions defined for the filtering step, were selected for the probes. An average and its standard deviation across the melting temperatures of 4,216 probes was 75.0±0.72° C. FIG. 17 shows the potentialities of cross-hybridization of 4,216 probes by means of the identities (%) between the probes and the most analogous gene fragments. The probing sequence 100% matches the gene, with which the probing sequence is intentionally hybridized. On the other hand, FIG. 17 shows the identities of between the probing probes and the gene sequences having a partial sequence of 80 bases except for the genes, with which the probing sequence is intentionally hybridized, out of 4,274 known rat genes registered in Unigene. According to FIG. 17, the identity between the probing sequence and the most analogous gene sequence is equal to or lower than 70%. The average and its standard deviation across the identities (%) between the probing sequence and the most analogous genes are 55.1±4.52%. This identity between the sequences simply indicates whether the alignments of ATGC match between the sequences and does not directly reflect the hybridization, a physicochemical phenomenon, as an index. FIG. 18 shows the potentialities of cross-hybridization for 4,216 probes by means of the melting temperature differences (° C.) from the most analogous genes and FIG. 19 shows the free energy differences (kcal/mol) from the most analogous genes, respectively. According to FIG. 18, the melting temperature differs by at least 20° C. or more from the most analogous partial sequence of 80 bases except for the genes, with which the probing sequence is intentionally hybridized, and those out of 4,274 known rat genes registered in Unigene. The average and its standard deviation across 4,216 genes is 75.6±24.3%. According to the typical melting curve drawn in hybridization between nucleic acids, it is expected that the difference of almost 10-15° C. in the melting temperature may prevent cross-hybridization from occurring. The difference of 20° C. or higher in the melting temperature may prevent almost completely cross-hybridization from occurring. According to FIG. 19, the stability (free energy) differs by at least 40 kcal/mol from the most analogous partial sequence of 80 bases except for the genes the genes, with which the probing sequence is intentionally hybridized, and those out of 4,274 known rat genes registered in Unigene. This means that that the stability in hybridization between the probes and the genes, with which the probing sequence is intentionally hybridized, is 40 kcal/mol higher than in that between the probing sequence and the most analogous gene fragment, suggesting that the former is stable by an average of 73.7±8.01 kcal than the latter.

The indexes of melting temperature difference and free energy difference are more preferable than the index of identity because they directly reflect the base sequences and hybridization, a physiochemical phenomenon. It may be known that the use of the prevent invention allows the user to determine the probing sequences having equal melting temperatures and sufficiently low potentialities of cross-hybridization from an aspect of practical use.

It was verified that 65 gene sequences, for which probing sequences were not determined with respect to 4,274 known rat genes, have more than one analogous genes having 80% or more homology between the gene sequences over its cDNA. Without shortening the probing sequence, probes, which do not cross-hybridize, cannot be determined. The problem exists in the genes themselves and the algorithm according to the prevent invention allows the user to determine all the determinable probing sequences.

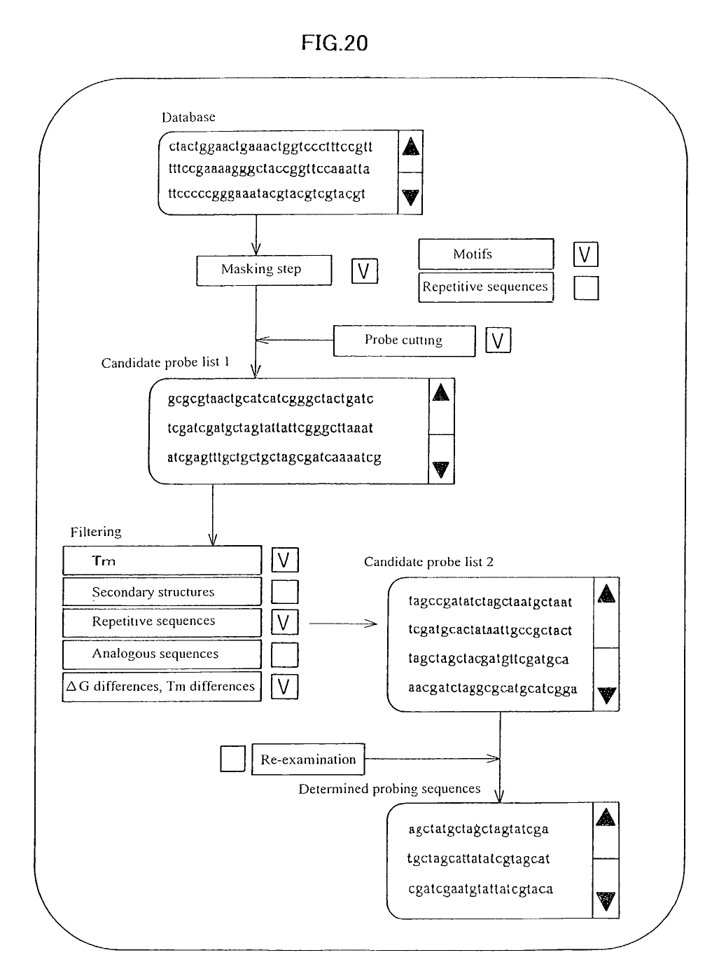

FIG. 20 shows an example of the interfaces explaining the whole process. First, the partial sequences of the candidate probe are selected from the genome or cDNA sequence database, the masking step is executed, in which the motif sites or the partial sequences containing repetitive sequence sites are excluded from the selected partial sequences, and the candidate probe list 1 is created. Next, based on the Tm values, secondary structures, repetitive sequences, analogous sequences, ΔG differences, and Tm differences, any nonconforming candidates unsuitable for the probes are filtered out, and the candidate probe list 2 is created. Next, the probing sequences are determined after the re-examination step.

The prevent invention thus provides the method for designing the probing DNA sequences to be fixed on the DNA probe array.

What is claimed is:

1. A probing sequence determination system comprising the steps of:
selecting partial sequences of candidate probes from a genome or cDNA sequence database;
masking the partial sequences containing motif sites and repetitive sequences for excluding from said selected partial sequences of candidate probes;
creating a primary candidate probe list registering the result of said masking step;
filtering given candidate probes for excluding from the primary candidate probe list; and
creating a secondary candidate probe list registering the result of said filtering step.

2. The probing sequence determination system according to claim 1, further comprising a probing-sequence cutting step for obtaining the candidate probes, which are partial sequences of bases with a second length shorter than a first length, from genome and cDNA sequences of bases with the first length among the candidate probes remaining in said masking step, wherein the primary candidate probe list registering the result of said probing-sequence cutting step is created.

3. The probing sequence determination system according to claim 1, wherein said filtering step further comprises a step of discarding the candidate probes base on either or both of melting temperature difference and free energy difference which are thermodynamic indexes.

4. The probing sequence determination system according to claim 3, further comprising a re-examination step of making selective analogous sequence search for splicing base sequences for selecting the sequences from said secondary candidate probe list when more than one transcribed product are produced from a single gene, and making analogous sequence search for base sequences for executing analogous sequence search for the base sequences registered in the database, to determine the probing sequences.

5. The probing sequence determination system according to claim 4, wherein said analogous sequence search executes analogous search for more than one species.

6. A probing sequence determination system comprising the steps of:
selecting the partial sequences for the candidate probes from the genome or cDNA sequence database;
masking the partial sequences containing given sites for excluding from the partial sequences of said selected candidate probes;
creating a primary candidate probe list registering the result of said masking step;
filtering the candidate probes for excluding from the primary candidate probe list based on either or both of melting temperature difference and free energy difference which are thermodynamic indexes; and
creating a secondary candidate probe list registering the result of said filtering step.

7. The probing sequence determination system according to claim 6, wherein said filtering step further comprises a step of discarding the candidate probes based on one or more than one of melting temperature, secondary structures, repetitive sequences, and analogous sequences.

8. The probing sequence determination system according to claim 7, wherein said secondary structures are the secondary structures of mini-hairpin structures.

9. The probing sequence determination system according to claim 7, wherein said repetitive sequence is one or more than one of a mini-hairpin sequence, simple repetitive sequence, and long repetitive sequence.
